(19) United States
(12) Patent Application Publication
(10) Pub. No.: US 2002/0111568 A1
(43) Pub. Date: Aug. 15, 2002

(54) Method for phased array ultrasonic transmission

(76) Inventor: Shmuel Bukshpan, Ramat Hasharon (IL)

Correspondence Address: DANIEL J SWIRSKY, PO BOX 2345, BEIT SHEMESH, 99544, IL

(21) Appl. No.: 09/829,069

(22) Filed: Apr. 10, 2001

Related U.S. Application Data
(63) Continuation-in-part of application No. 09/780,511, filed on Feb. 12, 2001.

(51) Int. Cl. A61B 8/00

(52) U.S. Cl. 601/2; 601/3; 600/437; 600/443; 600/468

(57) ABSTRACT

A method for phased-array ultrasonic transmission into a body wherein transmission from each element of the phased-array is implemented according to a firing order designed to generate a summated ultrasonic signal apparently originating from a virtual source outside of the body. The timing and order of firing of the phased-array elements is calculated from the relative proximities of each of the elements to the desired virtual source. The method allows for instantaneous ultrasonic localization of rapidly moving targets such as coronary thromboses.

### FIELD OF THE INVENTION

The present invention relates in general to ultrasonic interrogation and detection, and in particular to a method for achieving phased array ultrasonic detection of coronary thromboses.

### BACKGROUND OF THE INVENTION

Ultrasound is a diagnostic modality that is extensively used in a wide variety of medical fields, including the diagnosis of thromboses (blood clots) in blood vessels. As opposed to many other diagnostic modalities (such as x-rays, magnetic resonance imaging, nuclear scanning etc.) ultrasound is rapid, non-invasive and portable, making it highly suitable for use in urgent situations or in situations when access to more expensive modalities is limited. Furthermore, in addition to its utility as a diagnostic modality, ultrasound can be used therapeutically, as a means for achieving disruption of organic tissue (if the intensity of the ultrasound energy focused on the tissue is high enough). For example, blood clots have been shown to undergo thrombolysis when exposed to high intensity ultrasonic energy. Ultrasound can thus be used both to diagnose and to treat pathological thromboses in human beings. In this regard, the relevance of ultrasound to the diagnosis and treatment of coronary artery thrombosis is of particular interest.

It is known that coronary artery thrombosis is a leading cause of morbidity and mortality in the western world today. In this condition, a blood clot forms in a coronary artery, resulting in absent or inadequate blood flow to part of the heart muscle (myocardium). If adequate blood supply to the myocardium is not reestablished within minutes of onset of the thrombosis, the myocardium undergoes ischemia (cell damage), and later infarction (cell death). Effective treatment of coronary thrombosis thus depends on achieving dissolution of the thrombus (thrombolysis) as soon as possible after the onset of symptoms, and this is most commonly achieved by intravenous administration of a thrombolytic agent, such as tissue plasminogen activator (tPA).

Once a coronary thrombosis has been diagnosed, early thrombolysis is initiated as soon as possible. Ideally, this is done by medical or paramedical personnel at the point at which they first encounter the patient. Thus an ambulance team may initiate early thrombolysis prior to transporting a patient to the hospital, or hospital staff may initiate thrombolysis immediately upon arrival of a patient in the Emergency Room.

As the rapidity with which coronary blood flow is reestablished determines the long-term prognosis for the patient, there has been much interest in finding ways of augmenting or replacing standard early thrombolytic treatment protocols so as to achieve more rapid or more effective thrombolysis. For an early thrombolytic technique to be clinically useful, however, it is necessary that the technique be easily administered by the first medical personnel to encounter the patient (often an ambulance team or Emergency Room staff), at the point at which they first encounter the patient (often in the patient's home or in the hospital Emergency Room), over a short period of time. Ultrasound could be an ideal modality with which to achieve this goal, whereby the location of the coronary thrombosis could be determined ultrasonically, facilitating the performance of immediate ultrasonic thrombolysis by the same (or different) ultrasound transducers.

Implementation of ultrasonic thrombolysis requires that the exact spatial location of the thrombus in three dimensions be known, so as to facilitate accurate focusing of the ultrasonic energy on the thrombus. Three-dimensional ultrasonic spatial localization of a target in the human body can be achieved by utilizing a fixed array of ultrasound transducers (hereinafter also referred to as ultrasound “elements”), in which the spatial locations of the transducers relative to each other are fixed and known, to detect the target. By measuring the times-of-flight for ultrasound signals reflected off of the target and received by the ultrasound elements in the array, it is possible to calculate the distance between each element and the target, and therefore, by triangulation, the three-dimensional spatial location of the target in the body, relative to the fixed elements. By “phased array” is meant a fixed array of elements that can be activated to transmit ultrasound signals in a staggered, coordinated manner, and then receive the reflected signals

Conventional methods of utilization of phased arrays to detect and localize point targets, however, suffer from several deficiencies that negate their utility as means for localizing coronary thromboses:

1) Conventionally, each element of the array is activated in a sequential manner, such that the elements transmit one after the other. This allows the total time-of-flight between each element and the target to be calculated, and differences in times-of-flight for all the elements to be used to calculate the location of the target. Sequential firing of many elements, however, is time consuming. As coronary thromboses are located within the beating myocardium, coronary thromboses are constantly moving, and can be expected to change location during the lengthy process of sequential phased array firing, thus invalidating the acquired time-of-flight data for many of the elements.

2) Coronary thrombus detection is ideally achieved by analyzing the non-linear response (for example: the second harmonic component of the reflected signal, as will be explained below) of the target. To elicit a non-linear response by a target at an unknown location within a large anatomical area (such as the thorax), it is necessary to saturate the entire area under interrogation with an ultrasound signal of sufficient intensity. If only one ultrasonic element transmits at a time, the necessary minimum power at which the signal must be transmitted by each element is such that damage to skin tissue adjacent to the element is likely to occur. The necessary power of transmission necessary for a standard phased array to detect a coronary thrombosis in the thorax by means of second harmonic analysis techniques is thus prohibitive.

Thus, despite the proven efficacy of ultrasound as a method for achieving thrombolysis, the clinical application of this modality in the early treatment of coronary artery thrombosis has been inhibited by the technical difficulties involved in achieving rapid, real-time, three-dimensional ultrasonic localization of coronary thromboses by means of standard phased-array transmission techniques.

There is therefore a need for, and it would be highly advantageous to have, a method for phased-array ultrasound transmission which would allow both for more rapid firing of the elements in the array, and for transmission by each element at sufficiently low power as to prevent local tissue damage.

### SUMMARY OF THE INVENTION

The present invention provides a method for phased-array ultrasonic transmission suitable for facilitating thrombolysis of coronary artery thromboses by means of ultrasound energy. The method has many applications within the field of medical ultrasonic diagnosis and treatment beyond the achievement of coronary thrombolysis; however, to best explain and illustrate the method it will be described in the context of a method and device for the achievement of coronary thrombolysis.

In terms of the present invention, a thrombus-specific ultrasound contrast agent is injected intravenously in a patient suspected of having a coronary thrombosis. An array of ultrasound transducers positioned on the patient's chest then transmits ultrasound signals into the patient's thorax, and receives the reflected echoes. The ultrasound signals are transmitted concurrently in a synchronous but staggered manner, with an order and timing of firing specifically calculated to result in the generation of a summated wave which behaves as if it originated from a single apparent virtual source, usually behind, and distant from the phased-array. The firing sequence is sufficiently rapid that all of the array elements conclude transmitting before any begin receiving reflected echoes. This means that a full cycle of ultrasonic transmission and reception is completed within a period of time that is too short to have allowed significant movement of the target thrombus to take place. Furthermore, as all the elements in the array transmit almost concurrently, adequate saturation of the entire region under interrogation with an ultrasonic signal of sufficient power to elicit non-linear responses is achieved without the need for any single element to transmit at dangerously high power. Frequency or other temporal characteristics of the received echoes are then analyzed by a computer processor in real time, so as to instantly identify and spatially localize coronary artery thromboses. If a thrombosis is not identified, the location of the “virtual source” of transmission is moved, and the transmission process is repeated. If a thrombosis is identified, ultrasound signals of sufficient energy to cause thrombolysis are transmitted into the thorax by the phased-array transducers, focused on the identified spatial location of the thrombus.

The novelty of the present invention lies in implementing a firing order for ultrasonic transmission by elements of a phased-array in such a manner as to generate an ultrasonic wave having a single virtual point as its apparent source.

The present invention utilizes the observation that contrast bubbles exhibit a type of non-linearity that is different to that exhibited by native tissue. Typical bubble non-linearity in ultrasound reflection results in the generation of a second harmonic component that is relatively greater than that generated by native tissue, while the first harmonic components caused by bubble and native tissue non-linearity are relatively similar. Thus the ratio between the second and first transmitted harmonics of reflected echoes is notably higher for signals reflected off of a thrombus-specific ultrasound contrast agent than for signals reflected off of native organic tissues. Detection of a high ratio between the second and first transmitted harmonics thus indicates the presence of a thrombus in the ultrasonic field of interrogation. Determination of this ratio can be achieved instantaneously by a standard computer processor receiving data input from the transducer array, without the need for actual amplitude-based visualization of the tissue under interrogation. Furthermore, as the array is comprised of several transducers at different locations to each other on the thorax the processor can geometrically calculate the spatial source of the reflected signal having a high second to first harmonic ratio. As the calculations required to achieve diagnosis and localization of a thrombus by this method can be performed automatically and instantaneously, and as thrombolysis by means of the same transducer array can be initiated automatically by the computer processor (before the coronary artery has had time to move because of myocardial contraction), the method of the present invention allows for the rapid diagnosis and ultrasonic thrombolysis of coronary artery thromboses.

According to the teachings of the present invention there is provided, a method for achieving phased-array transmission into a body, including providing a plurality of ultrasound transmitters; selecting a location outside of the body, the location being noncoincident with the plurality of transmitters; transmitting an ultrasound signal from each of the transmitters into the body according to a time-sequence of transmission whereby a closest of the transmitters to the selected location transmits initially, and whereby others of the transmitters transmit after periods of time following the initial transmission that are proportional to the proximity of each of the other transmitters to the selected location. There is further provided an apparatus for achieving phased-array transmission into a body, including a sheet of material, the sheet being placeable on the body; a plurality of ultrasound transmitters for transmitting ultrasound signals into the body, the transmitters being fixedly located within the sheet, and the transmitters being oriented within the sheet such that the transmission is effected into the body; a layer of ultrasound coupling medium applied to a surface of the ultrasound transmitters, the layer being conformable to a contour of the body; and a processor for selecting a spatial location outside of the body, wherein the location is a virtual source for ultrasonic interrogation of the body, calculating a pattern of transmission for the ultrasound transmitters, wherein the pattern is proportional to the relative proximities of each of the transmitters to the selected spatial location, and effecting transmission of ultrasonic signals by the ultrasound transmitters according to the calculated pattern.

### BRIEF DESCRIPTION OF THE DRAWINGS

The invention is herein described, by way of example only, with reference to the accompanying drawings, wherein:

FIG. 1 is a graph of ratio of second harmonic to first harmonic components of a received ultrasound signal as a function of elapsed time;

FIG. 2 is a diagram of the principle components of a device capable of achieving ultrasonic coronary artery identification and localization in terms of the present invention;

FIG. 3 is a diagram of the geometry of a transducer crystal;

FIG. 4 is a diagram of transducer crystals in relation to the chest wall;

FIG. 5 is a diagram depicting the principle of virtual source generation; and

FIG. 6 is a block diagram detailing the process of ultrasonic thrombosis diagnosis and thrombolysis.

### DETAILED DESCRIPTION OF THE INVENTION

The present invention provides a method and system for ultrasonic phased-array transmission appropriate for achieving diagnosis and lysis of coronary artery thromboses.

The principles and operation of the method and system of the present invention may be better understood with reference to the drawings and the accompanying description.

It will be well known to one similar with the art that ultrasound signals transmitted into organic tissue at a given frequency (known as the “first harmonic”) are reflected off of a reflector in the organic medium as an ultrasound signal comprised of multiple frequency components. The central frequency component of the signal will be equal to the first harmonic, while additional components of the reflected signal will have frequencies equal to half of and to twice and possibly three times that of the first harmonic (termed the “half”, the “second” and “third” harmonics, respectively). This phenomenon of multiple harmonics in a reflected ultrasound signal occurs due to the non-linearity of organic tissue as an ultrasound conductive/reflective medium. It is further known that the second harmonic component of ultrasound signals reflected off of an ultrasound contrast agent (such as MRX-408, ImaRx Pharmaceutical Corp., Tucson, Ariz.) is relatively greater than the second harmonic components of ultrasound signals reflected off of an organic tissue reflector (such as muscle, blood vessels or thrombus). Thus, an ultrasound signal reflected off of an ultrasound contrast agent has a higher ratio of second harmonic to first harmonic frequency components than does a signal reflected off of an organic reflector. The “second to first harmonic ratio” in a reflected ultrasound signal is thus dependent on the nature of the reflecting object (be it a contrast agent or organic tissue), rather than on the amplitude of the reflected waves. Thus even a small quantity of ultrasonic contrast agent (generating reflected ultrasound signals of low amplitude) located within a highly echogenic organic tissue (generating multiple reflected ultrasound signals of high amplitude), can be ultrasonically identified by analyzing the second to first harmonic ratio of the received signals, even though the contrast agent itself cannot be discerned by standard ultrasonic imaging techniques (which construct a two-dimensional image based on the amplitudes of the received echo signals). If a thrombus-specific ultrasound contrast agent (which adheres specifically to intravascular thrombi) is injected intravenously, and thereafter ultrasonic interrogation of the subject's heart reveals a high second to first harmonic ratio in received ultrasonic signals, the presence of contrast agent in the heart (indicating the existence of a coronary artery, or other intracardiac, thrombosis) can be presumed.

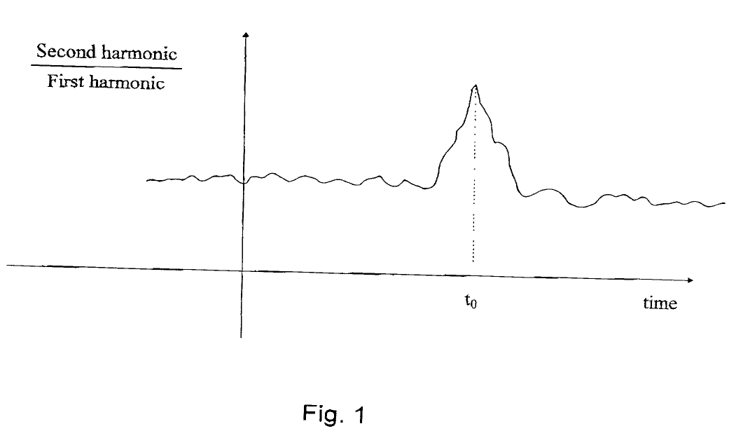

Turning now to FIG. 1, the result of ultrasonic interrogation of the heart of a patient (who was suspected of having a coronary artery thrombosis) is shown, after injection of Optison (a non-specific contrast agent, mimicking the mechanical properties of MRX-408) had been performed. The ultrasound signals were transmitted at a frequency of 1.5 MHz, and reflected signals were received by two crystals, one tuned to 1.5 MHz (being the first harmonic of the transmitted signal), and the second tuned to 3 MHz (being the second harmonic of the transmitted signal). The graph plots the ratio of second harmonic to first harmonic components of each received ultrasound signal as a function of the time elapsed since transmission of the signal. An increase in the second to first harmonic ratio can be seen at time to, indicating the presence of MRX-408 (most likely due to a coronary artery thrombosis) within the field of ultrasonic interrogation. As to (the time-of-flight for the signal with a high second to first harmonic ratio) is dependent on the distance between the contrast agent (e.g. MRX-408) reflector and the ultrasound receiver, additional receivers located at different locations on the patient's chest will each exhibit different values for to. If the geometric relationship between the three (or more) receivers on the patient's chest is known, an analysis of three such values for to allows the three-dimensional spatial location of the MRX-408 reflector, in relation to the ultrasound receivers, to be calculated by triangulation.

The values of to for each of multiple transceivers receiving echoes from a single reflective source can subsequently be used to generate a firing sequence for pulsed ultrasound signals transmitted from each of the multiple transceivers with such timing as to achieve maximal summation of the ultrasound energy at the site of the reflective source (namely, the MRX-408 contrast agent on a coronary thrombosis). If the power of the transmitted pulses is adequate, sufficient ultrasound energy can be focused on the thrombus to induce sonoparation and clot lysis, without damaging surrounding tissue.

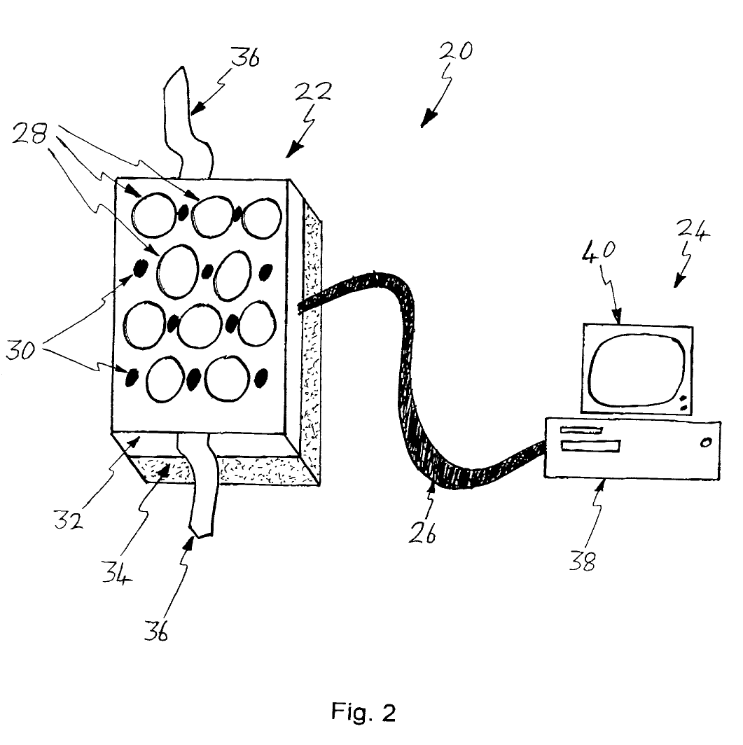

FIG. 2 is a diagram of the principle components of an ultrasonic thrombosis detection and lysis apparatus, hereinafter referred to as device 20, capable of achieving ultrasonic coronary artery thrombolysis based on the principles outlined in FIG. 1 above. As shown in the figure, device 20 is composed of a transducer pad 22, a control unit 24, and a cable 26 connecting transducer pad 22 and control unit 24. In an alternative embodiment (not shown in FIG. 2), control unit 24 is mounted directly onto transducer pad 22. Transducer pad 22 contains multiple ultrasound transducer crystals 28 and multiple ultrasound receiving crystals 30, arranged as a “phased-array”. In a preferred embodiment crystal 28 is a flat element (which transmits a beam whose divergence is limited by the ratio of the transducer size to the wavelength used) capable of transmitting at 1.5 to 3 MHz, and ranging in size between 1 and 3 mm. In this embodiment, a two dimensional array of flat elements of such size as to generate an effect of a convex (diverging beam) virtual source during the “diagnostic” phase of functioning of device 20, and a concave (converging beam) during the “therapeutic” phase of functioning of device 20, is used (see explanation below describing the “diagnostic” and “therapeutic” phases of functioning of the present invention). The specifications of crystal 28 are standard specifications well known to one familiar with the art.

In a preferred embodiment of the present invention transducer pad 22 includes sixty-four of transducer crystals 28 and sixty-four of receiving crystals 30. In an alternative embodiment, transducer pad 22 includes sixty-four of transducer crystals 28 and one hundred and twenty eight of receiving crystals 30. Transducer crystals 28 and receiving crystals 30 are embedded within a supporting sheet 32. Sheet 32 is a sheet of rigid material, typically plastic, which does not allow movement or translation between crystals 28 and 30 embedded within sheet 32 Crystals 28 and 30 are arranged in any manner in sheet 32, be it organized (for example, in a checkerboard arrangement relative to each other in sheet 32) or, preferably, be it completely random, or in any other arrangement within sheet 32 provided that the relative locations of each of crystals 28 and 30 to each other are fixed and known. Each of transducer crystals 28 and 30 are connected to cable 26. One surface of sheet 32 is affixed to a layer 34 of ultrasound coupling medium. Layer 34 is engineered as a plastic cushion enclosing aqueous ultrasonic coupling gel, and is sufficiently flexible as to conform to the contours of the chest. Crystals 28 and 30 are oriented in sheet 32 such that the transmitting/receiving surfaces of each of crystals 28 and 30 faces layer 34. Layer 34 serves as an interface between crystals 28 and 30 on the one hand, and the body of the patient, on the other. A strap 36 is affixed to either end of sheet 32. Sheet 32 can be held in place on the patient's body by straps 36. Several units of transducer pad 22 may be connected to each other in series (not shown) for use on a patient with a larger chest wall. Each of crystals 28 and 30 are connected to a computer processor 38 in control unit 24, by means of cable 26. Control unit 24 further includes a display 40. In alternative embodiments, sheet 32 can be held in place on the patient's body by the operator of device 20, or by other means.

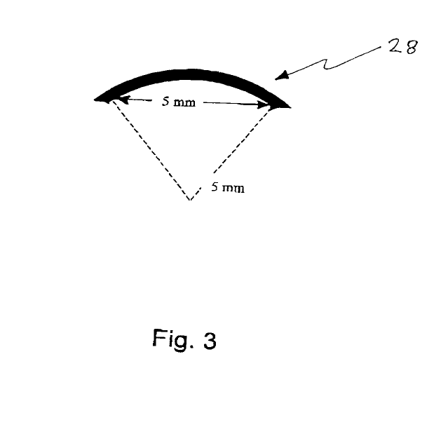

FIG. 3 is a diagram of the geometry of each of transducer crystals 28. As shown in FIG. 3, each crystal 28 is convex in shape, typically with a from radius of curvature, although crystal 28 may also be flat (not shown) or of any other shape, provided that the shape is known, without departing from the spirit of the invention. Each crystal 28 is operative to produce an omnidirectional ultrasound beam when activated. Transducer crystals 28 are typically tuned to transmit at 1.5 MHz. with receiving crystals 30 typically divided into three groups, a third being tuned to receive ultrasound signals at 1.5 MHz, a third of receiving crystals 30 being tuned to receive ultrasound signals at 3.0 MHz, and a third of receiving crystals 30 typically being tuned to receive ultrasound signals at 0.75 MHz.

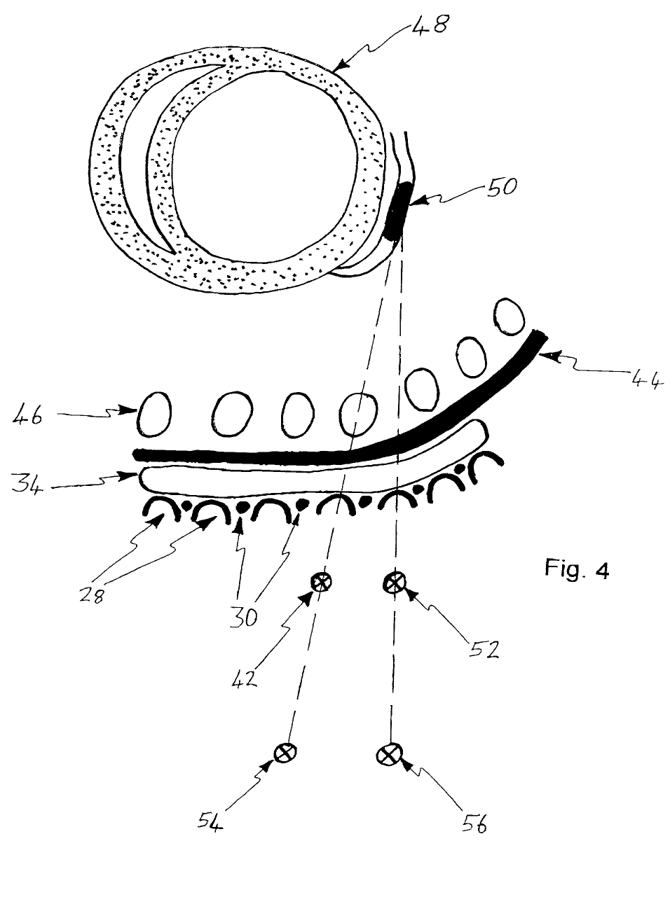

FIG. 4 demonstrates a preferred embodiment of the present invention. In the figure transducer crystals 28 and receiving crystals 30 are shown in position on the skin surface 44 of a patient's chest wall. Underlying skin surface 44 are ribs 46, the heart 48, and a coronary artery thrombosis 50. In this embodiment, during the thrombus identification process transducer crystals 28 all transmit ultrasound pulses as a “phased array” (i.e. in a synchronously staggered manner), such that summation of all the transmitted signals results in a convex (diverging) omnidirectional signal having as its apparent origin a “virtual source” located at a virtual point 42 outside of the patient's body. By changing the relative timing at which each of transducer crystals 28 transmit, the location of the virtual source can be moved. Ultrasound signal intensity at the site of thrombus 50 will depend on the position of virtual point 42, due to obstruction by ribs 46 of the signals transmitted by transducer crystals 28. Thus, for example, moving the virtual source from virtual point 42 to virtual point 52 in FIG. 4 will significantly enhance the signals reflected from thrombus 50. Since the ultrasound pressure amplitude at any point is dependant on the distance between that point and the source of the ultrasound signal (be it “real” or “virtual”), shifting the ultrasound source further away from skin surface 44 (e.g. from virtual point 42 to virtual point 54, or from virtual point 52 to virtual point 56) will result in lower ultrasonic intensities at skin surface 44 (for a given field intensity at the site of thrombus 50).

In terms of the preferred embodiment of the present invention, ultrasound signals are transmitted by transducer crystals 28 from a virtual source, and reflected echoes arc received by receiving crystals 30. In a preferred embodiment, the virtual source is located at a distance of 0.5 cm to 3 cm from transducer crystals 28, although it will be understood that the virtual source may be located at essentially any location outside of the patients body provided that the location of the virtual source is not coincident with the location of any of transducer crystals 28. The location of the virtual source can thus be said to be “noncoincident” with any of transducer crystals 28, meaning that the location of the virtual source does not coincide with the location of any of transducer crystals 28, and that there is a measurable distance between the two locations, typically at least 0.5 cm. The location of the virtual source is then changed by computer processor 38, in accordance with a preprogrammed set of virtual locations relative to sheet 32 stored in computer processor 38, and the process is repeated, until such time as a signal with a maximal second to first harmonic ratio is received by receiving crystals 30. The search for the signal having a maximal second to first harmonic ratio is initiated during an initial clot detection phase, when a very large area is scanned looking for the virtual location associated with a maximal echo. Once this position has been identified, the search is renewed after each thrombolysis sequence (lasting about 0.1 seconds) in a limited area, so as to compensate for minor changes in the position of sheet 32 (relative to the rib cage). Patient respiration or instability of the operator's hand holding sheet 32 could cause such changes in position.

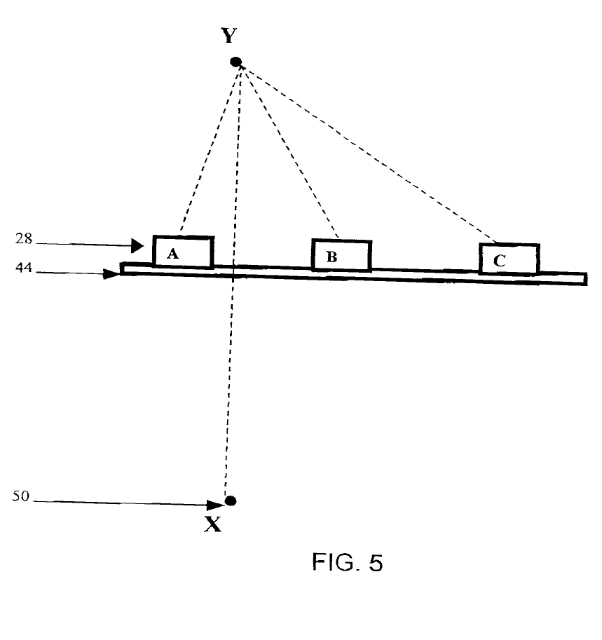

FIG. 5 is a diagram illustrating the principles of virtual source generation and target localization. The figure shows three transducer crystals 28 located at points “A”, “B”, and “C” on skin surface 44. A thrombus 50 is located at point “X” beneath the surface of skin 44. A virtual point in space “Y” is depicted behind transducer crystals 28. If the medium between point Y and points A, B, and C is homogeneous, the speed of ultrasound will be constant throughout the medium. In such a circumstance, the distance between point Y and any one of transducers 28 will be directly proportional to the time taken for an ultrasound signal to travel between the two points. Distances YA, YB, and YC can therefore represent time intervals with YA being the shortest and YC being the longest time intervals. If, non-concurrently, each of transducer crystals 28 transmit a single ultrasound pulse of identical waveform, such that transducer A fires first at time zero, transducer B fires second at a point in time equal to YB minus YA, and transducer C fires third at a point in time equal to YC minus YA, the three transmitted waves will summate to form a single diverging wave that behaves as would a wave transmitted from point Y. If this single wave then reflects off of thrombus 50 at point X and is received by each of transducer crystals 28, the relative times-of-flight for the received signals are proportional to the relative distances XA, XB, and XC. Note that distance YX is common to all the reflected signals, and can therefore be ignored. Transmission by transducer crystals 28 according to a time sequence based on the differences between distances XA, XB, and XC (in a manner analogous to that described above for distances YA, YB, and YC) will result in summation of the waves such that maximal ultrasonic energy is concentrated on thrombus 50 at point X.

In the preferred embodiment, device 20 functions as described below The operator of device 20 places pad 22 (typically one to five such pads, depending on the size and shape of the patient's thorax, held together by strap 36 or by the operator) on the chest of a patient in whom a coronary thrombosis is suspected, such that layer 34 is on the skin surface and that the patient's thorax is covered by pad 22. The operator injects a thrombus-specific ultrasound contrast agent (e.g. MX-408) intravenously into the patient. The operator then initiates operation of device 20 by means of control unit 24. Device 20 first performs a process of thrombus detection (the diagnostic phase). In this process, each of transducer crystals 28 transit a pulsed ultrasound signal into the chest of the patient in a timed sequence such that the effective beam produced has an apparent origin at a virtual source located distant from and behind transducer crystals 28. The location of the virtual source is determined by computer processor 38, and is the first of a set of virtual points constituting a predetermined empirical map of virtual points, which is stored in the memory of computer processor 38. Each pulsed ultrasound signal is typically transmitted with a central frequency of 1.5 MHz. Reflected ultrasound signals are then received by all of receiving crystals 30, some of which are tuned to the first harmonic (1.5 MHZ) some of which are tuned to the second harmonic (3 MHz), and others of which are tuned to the half harmonic (0.75 MHz). The received signals are quantified and the ratio of second to first harmonic components, or the ratio of the half to first harmonic components, in the received signals are calculated by computer processor 38. Each of transducer crystals 28 then transmit a pulsed ultrasound signal into the chest of the patient from a second virtual source located distant from and behind transducer crystals 28. The location of this second virtual source is determined by computer processor 38, and is the second of the set of virtual points constituting the map of virtual points stored in the memory of computer processor 38. Reflections from this signal are then received by receiving crystals 30 and processed as described above. This process is repeated sequentially for all of the virtual points stored in computer processor 38. If, once transmission from all of the virtual points has been completed, none of receiving crystals 30 has received a reflected signal having a significantly high second to first harmonic, as calculated by computer processor 38, an output on monitor 40 indicates to the operator of device 20 that no thrombosis was detected. A good signal is defined as a signal for which the ratio of second to first harmonics is in the range of 3:1 to 10:1, and preferably between 5:1 and 7:1. If, at any stage of transmission from the virtual points, any of receiving crystals 30 receive a reflected signal having a significantly high second to first harmonic ratio, as calculated by computer processor 38, an output on monitor 40 indicates to the operator of device 20 that a thrombosis has been detected. In this event, the operator may administer an intravenous thrombolytic agent (such as tPA) and then manually initiates a process (typically by pressing a button) of clot location and immediate lysis by device 20. In an alternative embodiment, the intravenous thrombolytic agent may be bound to a thrombus-specific ultrasound contrast agent.

The initial step in the process of clot localization and lysis (the therapeutic phase) is the same as the process of thrombus detection (the diagnostic phase) described above. Computer processor 38 calculates which of the virtual points generated reflected signals having the highest second to first harmonic ratio, and uses time-of-flight data from that transmission for subsequent calculations. In addition, whichever of receiving crystals 30 received a reflected signal over a predefined threshold amplitude are designated as being receivers unobstructed by the patient's ribs, and appropriate for utilization by device 20 in the process of thrombolysis. All other receivers are designated as being obscured by the patient's ribs, and are not utilized further by device 20. Computer processor 38 then calculates the time-of-flight (t₀) for each reflected ultrasound signal having a significant second to first harmonic ratio received by receiving crystals 30 (as demonstrated in FIG. 1 above). These time durations (less a preset constant which biases for the time-of-flight from the virtual point to the thrombus) are then used by computer processor 38 to calculate the spatial location of the diagnosed coronary thrombosis, and a timing sequence by which transducer crystals 28 may transmit a pulsed ultrasound signal so as to achieve thrombolysis. This timing sequence is such that omnidirectional ultrasound waves transmitted by transducer crystals 28 will summate at the calculated location of the coronary thrombosis, so as to achieve a focused field intensity of up to 190 W/cm², or a maximum negative pressure of 2.3 MPa, and induce thrombolysis. This pulsed ultrasound signal is typically a single pulse of 1-3 cycles (preferably 2 cycles). The repetition rate is dependent upon the range to the clot, as once a pulse has been fired, a second pulse cannot be fired until all echoes from the first pulse have subsided. Typical times between pulses are 100 and 300 microseconds (repetition frequencies between 3 and 10 KHz). Computer processor 38 then activates transducer crystals 28 according to the calculated timing sequence. Immediately after performing a “therapeutic” ultrasound transmission, system 20 repeats a “diagnostic” phase so as to detect if thrombus is still present, and if so, a repeat therapeutic cycle is performed. Several such cycles are performed until device 20 no longer detects evidence of thrombus. When this happens, monitor 40 indicates to the operator that thrombolysis has been performed. The process of thrombus identification and thrombolysis is achieved within several microseconds. The operator may then repeat the process of thrombosis detection and lysis by device 20 again, as needed.

The checkerboard arrangement of transducer crystals 28 and receiving crystals 30 relative to each other means that each one of transducer crystals 28 is surrounded by several of receiving crystals 30, in a spatially fixed manner. The differences in time-of-flight for the same received signal by each of “surrounding” receiving crystals 30 allows computer processor 38 to calculate the distance between each “central” transducer crystal 28 and the thrombus, and therefore the proper firing timings for all of transducer crystals 28.

FIG. 6 is a block diagram detailing the process of ultrasonic thrombosis diagnosis, and thrombolysis, as embodied in the present invention. The diagram describes a set of four transmitters 28 tuned to 1.5 MHz and two receivers 30 (one tuned to 1.5 MHz and the other tuned to the second harmonic, 3 MHz). For purposes of illustration only four of transducer crystals 28 and two of receiver crystals 30 are shown, although it should be understood that multiple units of crystals 28 and 30 function simultaneously as described in FIG. 5. Receivers 30 receive a reflected ultrasound signal 62 that had been transmitted 60 at a central frequency of 1.5 MHz by transmitters 28, in such a manner as to generate a virtual source for the resultant signal. Received signals 62 are then amplified 64 and undergo BPF (Band Pass Filter) antialiasing 66 and analog-to-digital conversion 68. The digital signals then pass through Band Pass Filters 70 tuned to either the first or second harmonic of transmitted signal 60. Band Pass Filters 70 select the frequency components of the signals equal to the relevant harmonic. The output signals from BPF 70 are input to envelope detectors 72 which detect and quantify the amplitude of the harmonic signals selected by BPF's 70. The quantified signals then undergo log compression 74, and the output log quantities are algebraically summated 76, with one quantity assigned a positive sign and the other quantity a negative sign, so as to generate a logarithm of the ratio of the two measured harmonics of signals 62. This value is input to a comparator 78, which compares the measured value with a threshold level 80 designated by the operator. When the measured value exceeds the threshold level a diagnosis of thrombosis is determined by processor 38. When processor 38 determines a diagnosis of thrombosis, processor 38 then determines which of transducer crystals 28 have acoustic access to the diagnosed thrombus. In the event that crystal 28 functions as a transceiver (switching from receiver electronic functioning to transmitter electronic functioning), this determination is made in the event that signal 62 had been received by crystal 28. Alternatively, in the event that crystal 28 functions as a transmitter only, this determination is made in the event that signal 62 had been received by those of receiving crystals 30 in the immediate vicinity of crystal 28, wherein the spatial relationship between crystals 28 and 30 in sheet 32 are fixed and known. Processor 38 then calculates an appropriate firing order for transducer crystals 28, based on the measured times-of-flight of signals 62 and the geometric relationships between crystals 28 in sheet 32, so as to focus the transmitted ultrasound energy on the diagnosed thrombus. Processor 38 then initiates functioning of signal source 80, which is a pulse generator functional to initiate ultrasound transmission by transducer crystals 28 in a “phased array mode”. Processor 38 then repeats the cycle of thrombosis diagnosis and thrombolysis according to a prescribed number of cycles or until a thrombus is not diagnosed.

Although the current invention has been described only with regard to coronary artery thrombolysis, it will be understood that there are many other applications for this invention. Thus, for example, the current invention may be used to facilitate carotid artery thrombolysis in cases of carotid artery stenosis due to plaque formation. In this condition, large pressure changes and high flow values occur at the carotid artery bifurcation, causing rapid movements of the arterial wall and necessitating a very rapid method of thrombus identification if immediate ultrasonic thrombolysis is to be achieved. Similarly, the current invention may be utilized for achieving thrombolysis in cases of deep venous thrombosis.

A further application of the current invention is in the ultrasonic detection of coronary artery stents. Stents are commonly placed in coronary arteries following balloon angioplasty, so as to prevent immediate restenosis. Frequently, however, re-stenosis occurs gradually, due to progressive endothelial tissue proliferation. Although ultrasound can be used to ablate such endothelial tissue, ultrasonic localization of the stent is difficult, due to the specular nature of ultrasonic reflection off of metal stents. Thus, when using a standard ultrasound transducer, the stent will often not be ultrasonically visible because the specularly reflected narrow beam signal does not return to the ultrasound transducer. However, use of an ultrasonic phased array, which covers a larger area of the thorax and is thus more likely to receive a narrow, specularly reflected signal, allows for the detection and spatial localization of coronary and other stents.

Although the present invention has been described with reference to the second harmonic of the transmitted ultrasonic signal, it should be understood that any other harmonic of the transmitted signal, for example the half harmonic, the third harmonic or the fourth harmonic (or indeed any harmonic which is different from the central frequency of transmission, the first harmonic), may be used instead of the second harmonic, or in addition to the second harmonic, without departing from the spirit of the present invention. Furthermore, although the present invention has been described with reference to frequency characteristics of the received ultrasornic signal, it should be understood that temporal characteristics of the received signal other than “frequency” (for example, characteristics generated by performing wavelet analysis or decomposition on the received ultrasound signals rather than Fourier analysis) may be analyzed in a similar manner to that described above for frequency characteristics, so as to rapidly diagnose a coronary thrombosis, without departing from the spirit of the present invention. By “temporal characteristic” is meant any ultrasound characteristic or parameter that is a function of time, and requires, of necessity, a time axis for its graphic depiction. “Temporal characteristics” are thus to be contrasted with those ultrasound characteristics or parameters which can be quantified in terms independent of time, for example amplitude and power.

There has thus been described a method for achieving ultrasonic phased-array transmission into a subject's chest with sufficient intensity as to elicit non-linear responses throughout the area under interrogation, which is sufficiently rapid as to allow for the accurate and immediate spatial localization of coronary thromboses, and which can be achieved utilizing sufficiently low power transmission from each array element as to allow transmission to be safely achieved by a phased-array placed directly on, or in close proximity to, the subject's skin.

It is appreciated that one or more of the steps of any of the methods described herein may be omitted or carried out in a different order than that shown, without departing from the true spirit and scope of the invention.

While the present invention as disclosed herein may or may not have been described with reference to specific hardware or software, the present invention has been described in a manner sufficient to enable persons of ordinary skill in the art to readily adapt commercially available hardware and software as may be needed to reduce any of the embodiments of the present invention to practice without undue experimentation and using conventional techniques.

While the present invention has been described with reference to one or more specific embodiments, the description is intended to be illustrative of the invention as a whole and is not to be construed as limiting the invention to the embodiments shown. It is appreciated that various modifications may occur to those skilled in the art that, while not specifically shown herein, are nevertheless within the true spirit and scope of the invention.

What is claimed is:

1. A method for achieving phased-array transmission into a body, comprising:
a) providing a plurality of ultrasound transmitters;
b) selecting a location outside of the body, said location being noncoincident with any of said plurality of transmitters; and
c) transmitting an ultrasound signal from each of said transmitters into the body according to a time-sequence of transmission whereby a closest of said transmitters to said selected location transmits initially, and whereby others of said transmitters transmit after periods of time following said initial transmission that are proportional to the proximity of each of said other transmitters to said selected location.

2. The method of claim 1, wherein said plurality of transmitters includes a phased-array of transmitters having fixed locations relative to each other.

3. The method of claim 1, wherein said location is a virtual source from which it is desired to ultrasonically interrogate the body.

4. The method of claim 1, wherein said transmitted ultrasound signals undergo summation within the body to produce an ultrasound signal having as its apparent origin said selected location.

5. The method of claim 1, further comprising the step of d) repeating steps b and c for a plurality of locations.

6. Apparatus for achieving phased-array transmission into a body, comprising:
a) a sheet of material, said sheet being placeable on the body;
b) a plurality of ultrasound transmitters for transmitting ultrasound signals into the body, said transmitters being fixedly located within said sheet, and said transmitters being oriented within said sheet such that said transmission is effected into the body;
c) a layer of ultrasound coupling medium applied to a surface of said ultrasound transmitters, said layer being conformable to a contour of the body; and
d) a processor for:
i) selecting a spatial location outside of the body, wherein said location is a virtual source for ultrasonic interrogation of the body,
ii) calculating a pattern of transmission for said ultrasound transmitters, wherein said pattern is proportional to the relative proximities of each of said transmitters to said selected spatial location, and
iii) effecting transmission of ultrasonic signals by said ultrasound transmitters according to said calculated pattern.

7. The apparatus of claim 6, wherein said sheet includes plastic.

8. The apparatus of claim 6, wherein said transmitters are arranged in a checkerboard pattern relative to each other in said sheet.

9. The apparatus of claim 6, wherein said pattern is a time-sequence of transmission whereby a closest of said transmitters to said selected location transmits initially, and whereby others of said transmitters transmit after periods of time following said initial transmission that are proportional to the proximity of each of said other transmitters to said selected location.
